(19) United States
(12) Patent Application Publication
(10) Pub. No.: US 2018/0199583 A1
(43) Pub. Date: Jul. 19, 2018

(54) DAIRY PRODUCT

(71) Applicant: GODO SHUSEI CO., LTD., Chuo-ku (JP)

(72) Inventors: Junki OGASAWARA, Matsudo-shi (JP); Hirofumi HORIGUCHI, Matsudo-shi (JP)

(73) Assignee: GODO SHUSEI CO., LTD., Chuo-ku (JP)

(21) Appl. No.: 15/741,906

(22) PCT Filed: Jul. 5, 2016
(86) PCT No.: PCT/JP2016/069859

§ 371 Date: Jan. 4, 2018

(30) Foreign Application Priority Data
Jul. 6, 2015 (JP) 2015-135591

(51) Int. Cl. A23C 9/127; A23C 19/032; A23C 19/055; C12N 9/58

(52) U.S. Cl. CPC A23C 9/127; C12N 9/58; A23C 19/055; A23C 19/0325

(57) ABSTRACT

A novel dairy product that has a certain solidity and a favorable flavor is provided. A dairy product, including: a milk-derived raw material containing casein; and a protein consisting of an amino acid sequence having 90% or more identity to the amino acid sequence represented by SEQ ID NO: 1.

### TECHNICAL FIELD

The present invention relates to a dairy product with new texture, and a method for producing the dairy product.

### BACKGROUND ART

Dairy products include one in a liquid state and one in a solid state. As the dairy product in a solid state, cheese, fermented milk (for example, yogurt), and the like have been known. Yogurt becomes solid as a result of lactic acid fermentation. Meanwhile, cheese becomes solid by action of rennet (enzyme made in mammalian stomach) that is milk-coagulating enzyme, on casein (Patent Literature 1). In addition, as the rennet, microbially derived rennet, and genetically modified rennet are also known (Patent Literatures 1 and 2).

### CITATION LIST

### Patent Literature

### Patent Literature 1: JP 2004-33093 A

### Patent Literature 2: JP hei 02-18834 B

### SUMMARY OF INVENTION

### Technical Problem

However, there is a problem that a dairy product coagulated with rennet generates a bitter taste. In addition, yogurt by lactic acid fermentation has insufficient solidity in many cases, and a thickening agent and a gelling agent may be mixed in commercially available yogurt by taking into consideration the shape retention during distribution and storage.

Accordingly, development of a novel dairy product that has a certain solidity and a favorable flavor is desired.

### Solution to Problem

Herein, as a result of extensive studies to find a novel milk-coagulating component to replace rennet, the present inventors have found that a protein and an analog thereof, which are predicted to be produced by a microorganism belonging to the genus Trichoderma, and which is nothing more than its amino acid sequences are predicted, have an excellent milk-coagulating action, and in particular, fermented milk obtained by the fermentation together with lactic acid bacteria or yeast has a cheese-like solidity, and becomes a dairy product having no bitterness but having a favorable flavor, and thus have completed the present invention.

That is, the present invention provides the following [1] to [8].

[1] A dairy product, including: a milk-derived raw material containing casein; and a protein consisting of an amino acid sequence having 90% or more identity to the amino acid sequence represented by SEQ ID NO: 1.

[2] The dairy product according to [1], wherein the protein is a protein derived from a microorganism belonging to the genus Trichoderma.

[3] The dairy product according to [1] or [2], further including lactic acid bacteria or yeast.

[4] The dairy product according to any one of [1] to [3], wherein the dairy product is fermented milk or cheese.

[5] The dairy product according to any one of [1] to [4], wherein the dairy product has a breaking stress in a two-bite method of 5,500 Pa or more, and a breaking adhesion of 800 J/m³ or more.

[6] The dairy product according to any one of [1] to [5], substantially not including a coagulation component selected from the group consisting of a thickening agent, a stabilizer, a gelling agent, and an adhesive paste.

[7] A method for producing a dairy product, including: adding a protein consisting of an amino acid sequence having 90% or more identity to the amino acid sequence represented by SEQ ID NO: 1, and lactic acid bacteria to a milk-derived raw material containing casein; and fermenting a mixture of the protein and the lactic acid bacteria with the milk-derived raw material.

[8] A milk-coagulating agent for a dairy product, including a protein consisting of an amino acid sequence having 90% or more identity to the amino acid sequence represented by SEQ ID NO: 1.

### Advantageous Effects of Invention

The dairy product of the present invention has a cheese-like solidity and imparts a favorable flavor, and is useful as a dairy product with new texture. By variously adjusting the reaction conditions between the specific protein used in the present invention and the milk-derived raw material, the dairy product of the present invention can be produced so as to have a solidity in a range of being harder than that of the conventional yogurt and softer than that of the conventional cheese.

### BRIEF DESCRIPTION OF DRAWINGS

FIG. 1 shows the temperature dependency of GODO-TCF derived protease (SEQ ID NO: 1).

FIG. 2 shows the temperature stability of GODO-TCF derived protease (SEQ ID NO: 1).

FIG. 3 shows the pH stability of GODO-TCF derived protease (SEQ ID NO: 1).

FIG. 4 shows the pH dependency of GODO-TCF derived protease (SEQ ID NO: 1).

FIG. 5 shows the effect of each enzyme at pH 5.5 to casein.

FIG. 6 shows the effect by GODO-TCF derived protease (SEQ ID NO: 1) at pH 7.2.

FIG. 7 shows the coagulability of pH-adjusted milk by GODO-TCF. Top: heat treatment, Medium: GODO-TCF 1% addition, and Bottom: GODO-TCF 10% addition.

FIG. 8 shows the changes with time of pH in yogurt during the fermentation of lactic acid bacteria are shown. (Each of arrows shows a point where the surface does not move when the tube is tilted.)

FIG. 9 shows the results of breaking strength analysis.

FIG. 10 shows the results of texture analysis under non-breaking conditions.

FIG. 11 shows the results of texture analysis under breaking conditions.

### DESCRIPTION OF EMBODIMENTS

As the protein consisting of an amino acid sequence having 90% or more identity to the amino acid sequence represented by SEQ ID NO: 1, which is used in the dairy product of the present invention, a protein derived from a microorganism belonging to the genus Trichoderma is preferred. More specifically, for example, a functionally unknown predicted protein derived from Trichoderma reesei QM6a (available fungus described in National Center for Biotechnology Information (NCBI)) (100% identical to the amino acid sequence represented by SEQ ID NO: 1), a protein contained in a commercially available cellulase GODO-TCF derived from Trichoderma reesei (100% identical to the amino acid sequence represented by SEQ ID NO: 1), a virtual protein TRIVIDRAFT_215801 derived from Trichoderma virens Gv29-8 (available fungus described in NCBI) (94% identical to the amino acid sequence represented by SEQ ID NO: 1: SEQ ID NO: 2), a vacuolar protein A derived from Trichoderma atroviride (92% identical to the amino acid sequence represented by SEQ ID NO: 1: SEQ ID NO: 3), and a vacuolar protein derived from Trichoderma aureoviride (93% identical to the amino acid sequence represented by SEQ ID NO: 1) can be mentioned.

A more preferred protein is a protein having 92% or more identity to the amino acid sequence represented by SEQ ID NO: 1, an even more preferred protein is a protein having 93% or more identity to the amino acid sequence represented by SEQ ID NO: 1, a still more preferred protein is a protein having 94% or more identity to the amino acid sequence represented by SEQ ID NO: 1, and a yet more preferred protein is a protein having 95% or more identity to the amino acid sequence represented by SEQ ID NO: 1.

The protein used in the present invention may be extracted from a microorganism belonging to the genus Trichoderma, or may be produced by a gene recombination technique. Note that in the protein, as long as a milk-coagulating action is contained, a protein to which a sugar is bound is also included.

The protein used in the present invention has caseinolytic activity, therefore, has protease activity. Among caseins, the degradation activity to κ-casein is high the degradation activity to α-casein and β-casein is weaker than the degradation activity to κ-casein.

The protein degrades the κ-casein contained in a milk-derived raw material with a slight addition amount. According to this, α-casein (including degradation products) and β-casein (including degradation products) aggregate, and the solidity of a dairy product increases.

As the protein, a purified protein may be used, and, for example, a culture (including a culture solution) of the microorganism belonging to the genus Trichoderma containing the protein, and an extract of the culture can be used. For example, the above-described cellulase GODO-TCF may be used as it is.

As the milk-derived raw material containing casein, which is used in the dairy product of the present invention, as long as casein is contained, for example, a liquid material or a powder material of cow milk, goat milk, sheep milk, horse milk, breast milk, or the like can be mentioned. In a case where the powdered milk is used, one mixed with, for example, water may be used. Note that in milk, raw milk, low-fat milk, fat-free milk, and processed milk are also included.

The content of the protein contained in the dairy product of the present invention may be a content with which the milk-coagulating activity is exerted. Whether or not the milk-coagulating activity is exerted varies depending on, for example, the kind of the milk-derived raw material to be used, the amount of the casein contained in the milk-derived raw material, and the production conditions (for example, reaction time, reaction temperature and reaction pH). For this reason, although it cannot be said sweepingly, as to the content of the protein, for example, if the milk-coagulating activity (caseinolytic activity) of the protein when the protein is added to a milk-derived raw material is in the range of 20 U to 300 U per 100 g of the milk raw material, a dairy product having an unprecedented solidity in the past is easily obtained.

The presence or absence of the casein degradation can be confirmed by analyzing the obtained dairy product by, for example, sodium dodecyl sulfate-polyacrylamide gel electrophoresis (SDS-PAGE).

The acid protease activity of the protein contained in the dairy product of the present invention is preferably 3,000 U or less per 100 g of the milk raw material.

When the activity exceeds the level, flavor of the dairy product may be deteriorated.

It is preferred that the dairy product of the present invention further contain lactic acid bacteria or yeast from the viewpoint of the flavor and the texture. In the dairy product used the protein in combination with lactic acid bacteria, the solidity is increased as compared with that of the conventional fermented milk, therefore, it is more preferred that the dairy product of the present invention contain lactic acid bacteria.

Herein, as the Lactic acid bacteria, those belonging to the genus lactic acid bacteria, the genus Bifidobacterium, the genus Enterococcus, the genus Lactococcus, or the genus Leuconostoc can be used.

The content of lactic acid bacteria in the dairy product of the present invention is not particularly limited, however, is preferably 1,000,000/1 mL or more, more preferably 5,000,000/1 mL or more, and even more preferably 10,000,000/1 mL or more.

In the dairy product of the present invention, if necessary, in addition to a sweetener such as syrup, various other food materials, for example, optional components such as various sugars, an emulsifier, and various vitamin preparations can be mixed. Specifically, a sugar such as sucrose, glucose, fructose, palatinose, trehalose, lactose, xylose, and maltose; a sugar alcohol such as sorbitol, xylitol, erythritol, lactitol, palatinit, reduced sugar syrup, and reduced maltose syrup; a high-intensity sweetener such as aspartame, thaumatin, sucralose, acesulfame K, and stevia; an emulsifier such as a sucrose fatty acid ester, a glycerine fatty acid ester, a polyglycerol fatty acid ester, a sorbitan fatty acid ester, and lecithin; milk fat such as cream, butter, and sour cream, an acidulant such as citric acid, lactic acid, acetic acid, malic acid, tartaric acid, and gluconic acid; various vitamins such as vitamin A, vitamins B, vitamin C, and vitamins E; minerals such as calcium, magnesium, zinc, iron, and manganese; and various flavors can be mixed.

The dairy product of the present invention has sufficient solidity due to the milk-coagulating action of the protein, therefore, it is not required to contain a coagulation component such as a thickening agent, a stabilizer, a gelling agent, and an adhesive paste, and it is preferred not to substantially contain such a coagulation component.

The dairy product of the present invention can be produced in accordance with a conventional method for producing fermented food by mixing the milk-derived raw material and the protein, and milk-coagulating the resultant preparation, or by mixing the milk-derived raw material, the protein, and lactic acid bacteria or yeast. As a method for producing fermented food, a method in which the milk-derived raw material and the protein are mixed, lactic acid bacteria or yeast is inoculated into the resultant preparation and the inoculated preparation is cultured, and after homogenization of the cultured preparation, for example, an optional component such as a syrup liquid, and a flavor are added can be mentioned.

The dairy product obtained according to the present invention has a specific texture of having a solidity that is higher than the solidity of the yogurt in a solid state, and further has favorable flavor without having, for example, a bitter taste, in spite of containing no thickening agent or the like. Such a texture is a specific effect due to the protein described above.

In the dairy product of the present invention, preferably, the breaking stress is 5,500 Pa or more and the breaking adhesion is 800 J/m³ or more in a two-bite method, in view of the texture, and further the breaking stress is more preferably 5,500 to 12,000 Pa, and the breaking adhesion is more preferably 800 to 1,200 J/m³.

As the preferred form of the dairy product of the present invention, fermented milk, cheese, ice cream, ice milk, and lacto-ice can be mentioned, but fermented milk and cheese are more preferred. As the fermented milk, a form of solid-type yogurt is particularly preferred.

The protein acts on casein and shows a milk-coagulating action, therefore is useful as a milk-coagulating agent for a dairy product.

### EXAMPLES

Next, the present invention will be described by way of examples.

### Production Example 1

(1) (Separation of Protease from GODO-TCF)

100 mL of GODO-TCF (mutant of Trichoderma reesei QM6a, manufactured by GODO SHUSEI CO., LTD.) was concentrated to about 50 mL with a UF pencil-type module API-0013 (molecular weight cutoff of 6,000), and into the concentrated preparation, 150 mL of a 20 mM MES buffer solution (pH 6.0) was added. This procedure was repeated four times.

The obtained concentrate was applied to a Giga Cap Q-650M (TOYOPEARL) column equilibrated with a 20 mM MES buffer solution (pH 6.0), and a fraction containing the milk-coagulating activity eluted with a linear gradient of 0-0.5 M NaCl was recovered. Into the recovered active fraction, ammonium sulfate (hereinafter, referred to as AS) was added so that the final concentration is 0.5 M, and then the resultant preparation was applied to a Butyl-650 M (TOYOPEARL) column equilibrated with a 20 mM MES buffer solution (pH 6.0) containing 0.5 M AS, and eluted with a linear gradient of 0.5-0 M AS. Then, the active fraction was recovered, and concentrated with Amicon Ultra™-30K (molecular weight cutoff of 30 kDa) (4,000×g, 120 minutes), and then the resultant preparation was applied to a Hiprep 16/60 Sephacryl S-200 HR column (GE Healthcare) equilibrated with a 20 mM MES buffer solution (pH 6.0) containing 0.15 M NaCl, and the active fraction was recovered.

When the active fraction was subjected to SDS-PAGE, single band was confirmed at around 33 kDa.

### (2) Amino Acid Sequencing of GODO-TCF Derived Protease

The activated fraction eluted by Butyl-650 M column chromatography in the purification process described in (1) was subjected to SDS-PAGE, and a target enzyme band at about 33 kDa was excised, and then from a sequence of a peptide fragment, gene sequence information of a predicted protein [Trichoderma reesei QM6a] having high homology of the amino acid sequence from the NCBInr database using a Mascot seach program was obtained. On the basis of the obtained gene information, PCR primers 5′-3′ (Foward: SEQ ID NO: 4), and 5′-taggactgtcttccgctctcaaactgc-3′ (Reverse: SEQ ID NO: 5) were designed, and using a genomic DNA of a Trichoderma reesei QM9414 strain as a template, the target gene was amplified with EX Taq (TaKaRa). The amplified DNA fragment of about 1,600 bp was excised, and purified using FastGene Gel/PCR Extraction (NIPPON Genetics Co, Ltd), and then the DNA sequence information was obtained, and the amino acid sequence was determined by a translation program. The obtained amino acid sequence was shown as SEQ ID NO: 1. The amino acid sequence was 100% identical to the amino acid sequence of Trichoderma reesei QM6a.

### (3) Other Properties of GODO-TCF Derived Protease

### (Method)

An enzyme sample of the eluted active fraction of Butyl-650M during the purification process described in (1) was used. The temperature dependency was determined using chymosin activity at 20, 30, 40, 45, 50, 55, 60, or 65° C. as an indicator. Measurement of chymosin activity was performed by a method in which the International Committee for Standardization in Hematology was partially modified. The substrate solution was prepared by dissolving low-heat low-fat spray dried milk powder at a concentration of 11 g/100 mL, adding CaCl₂ so as to have a final concentration of 3 mM, and then adjusting the pH to 5.5 with 1 N hydrochloric acid. Into 0.1 mL of the enzyme solution preincubated at a measurement temperature, 5 mL of the substrate solution preincubated in the same manner was added, and the resultant preparation was stirred so as not to foam. After that, a test tube was tilted around every one minute to check whether coagulated milk adheres to the wall surface, while conducting the reaction at each temperature, and the time when the coagulated milk adheres for the first time was measured. The measurement was performed at the same time as the measurement of a reference standard with a known measurement value, and each milk-coagulating time was examined and the activity level was calculated.

As to the temperature stability, the enzyme solution was incubated at 4, 20, 30, 40, 50, 60, or 70 for 2 hours, and then the remaining chymosin activity after diluting the incubated preparation by 2 times with a 100 mM MES buffer (pH 5.5, r.t.) was measured in accordance with the activity measurement method described above.

As to the pH stability, the enzyme solution was diluted by 3 times with each buffer at pH 3.0, 3.5, 4.0, 4.5, 5.0, 5.5, 6.0, 6.5, 7.2, 7.5, 8.0, 8.5, or 9.0 (pH 3.0 to 6.0:0.2 M citric acid-Na citrate buffer, pH 6.0 to 8.0:0.2M phosphoric acid-Na phosphate buffer, and pH 7.2 to 9.0:Tris-HCl buffer), and the diluted preparation was left to stand at 4° C. for 6 hours. Then, the resultant preparation was diluted by 2 times with a 100 mM MES buffer (pH 5.5, r.t.), and the remaining chymosin activity was measured in accordance with the activity measurement method described above.

The pH dependency was evaluated by a method using a releasing activity of acid-soluble peptides. As to the substrate solution, 1.2 g of milk casein (CALBIOCHEM) was weighed, and to the milk casein, 20 mL of distilled water and 20 mL of a 0.1 M Tris solution were added and dissolved, and then the resultant preparation was heated at 60° C. for 10 minutes in a boiling bath. After that, into the heated preparation, 100 mL of each 500 mM buffer (pH 3.0 to 6.0:0.2 M citric acid-Na citrate buffer, pH 6.0 to 8.0:0.2 M phosphoric acid-Na phosphate buffer, and pH 7.2 to 9.0:Tris-HCl buffer), was added, and each pH was adjusted with 1 N hydrochloric acid or sodium hydroxide, and then the resultant preparation was filled up to 200 mL with distilled water to perform the preparation. In the reaction, 0.5 mL of the enzyme solution was preincubated at 50° C. for 3 minutes, 2.5 mL of the substrate solution was added to the preincubated preparation, and the resultant preparation was thoroughly stirred and reacted at 50° C. for 10 minutes. After that, 1.8 g of trichloroacetic acid, 11.45 g of acetic anhydride, and 21 mL of acetic acid were mixed, into the mixture, 2.5 mL of a precipitation reagent that had been adjusted to 1 L with distilled water was added, and the resultant preparation was left to stand at room temperature for about 5 minutes. After that, a filtrate obtained by filtering the resultant preparation through a ADVANTEC No. 4A filter paper was measured for the absorbance in 275 nm, and the activity level was calculated using the enzyme level at which acid-soluble low molecular weight degradation products corresponding to 1 μg of tyrosine per minute are generated as 1 unit.

### (Results)

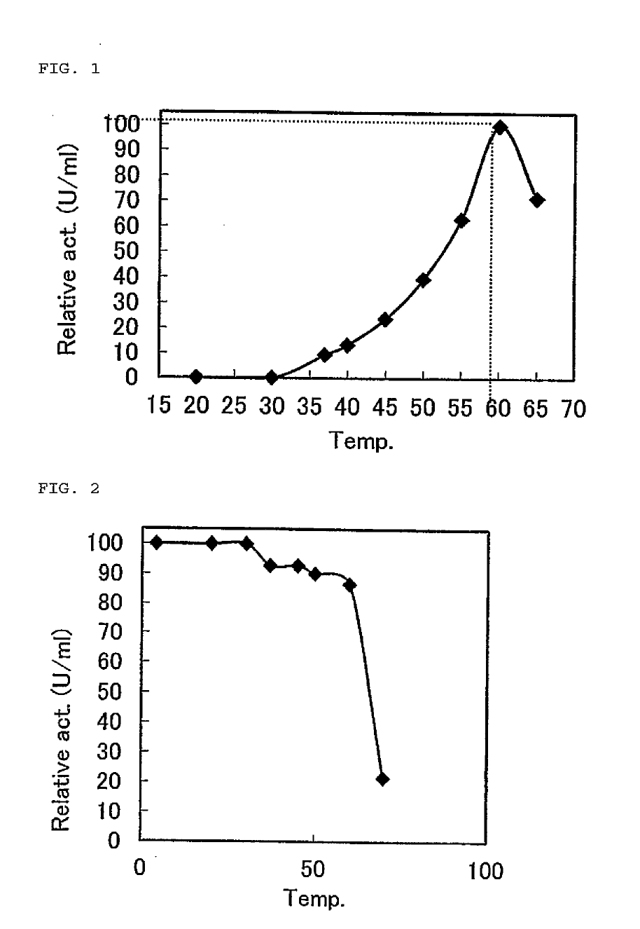

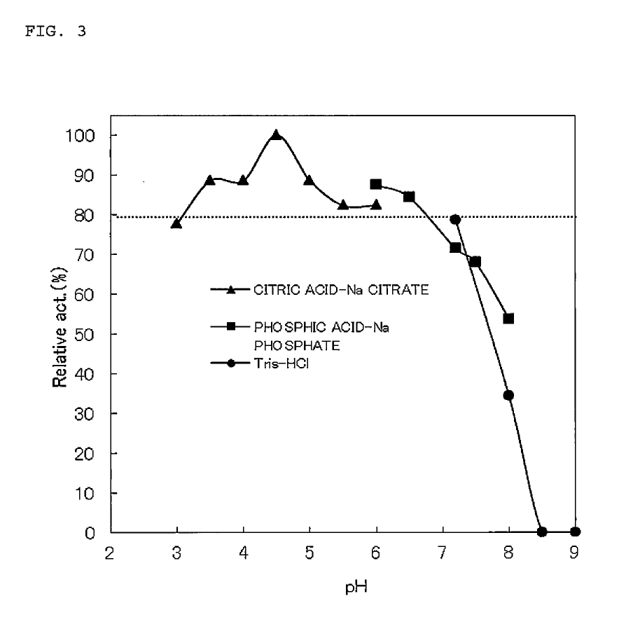

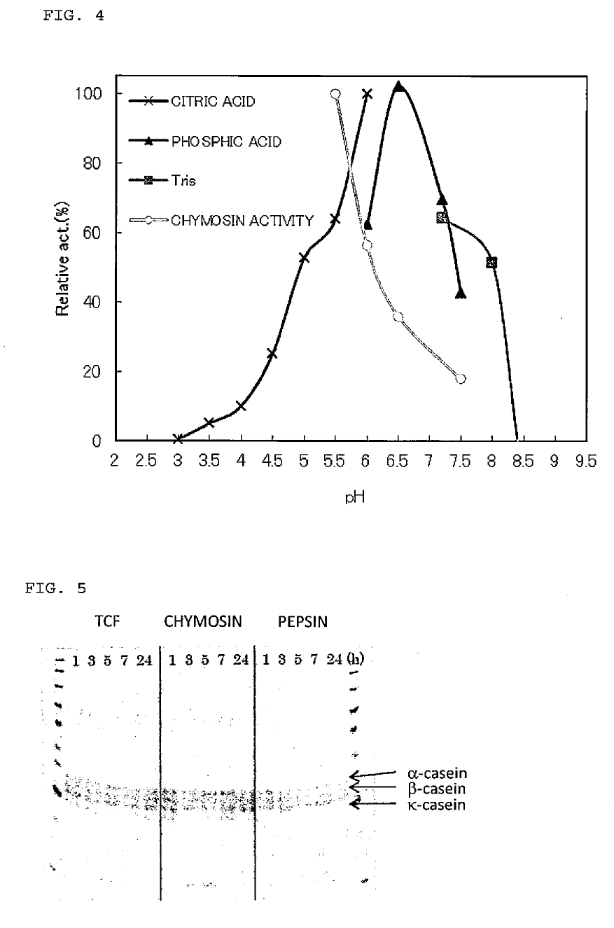

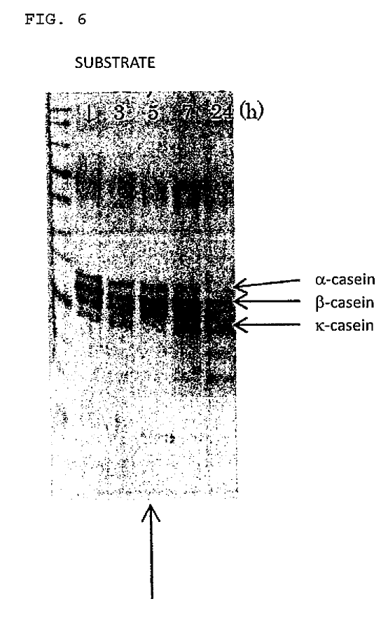

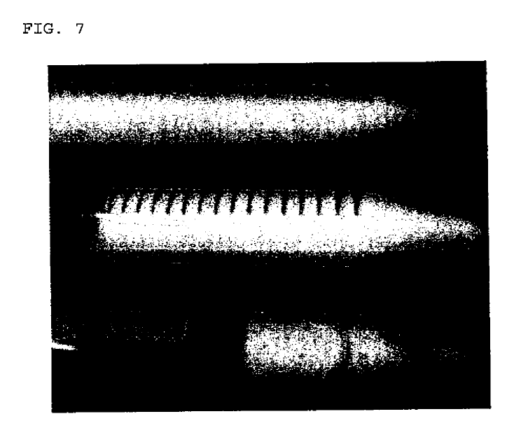

(1) The temperature dependency of the GODO-TCF derived protease (the protease represented by SEQ ID NO: 1) is shown in FIG. 1, the temperature stability of the GODO-TCF derived protease is shown in FIG. 2, the pH stability of the GODO-TCF derived protease is shown in FIG. 3, and the pH dependency of the GODO-TCF derived protease is shown in FIG. 4. As shown in FIGS. 1 to 4, the GODO-TCF derived protease showed the maximum activity at about 60° C., and maintained the residual activity of 80% or more at pH 3.5 to 6.5 and up to 60° C. In the pH dependency, it can be assumed that the optimum pH of chymosin activity is on the acidic side from 5.5, whereas it can be understood that the acid-soluble peptide releasing activity is in the vicinity of pH 6.5.
(2) The effect of GODO-TCF derived protease to casein at pH 5.5, and the effect of the chymosin and pepsin contained in rennet on casein were examined. The effect of GODO-TCF-derived protease to casein at pH 7.2 was examined. The results are shown in FIGS. 5 and 6.

As a result, it was suggested that the GODO-TCF derived protease has high specificity for κ-casein at pH 5.5 (FIG. 5), whereas further acts to α and β-caseins in the vicinity of pH 7.2 (FIG. 6). It was confirmed that chymosin has high specificity for κ-casein, and pepsin has a thinner band as a whole. It was found that when comparing the GODO-TCF derived protease with the other two proteases, the specificity of the GODO-TCF milk coagulating protease was κ-casein>α-casein.

### Example 1

The pH was adjusted to 5.9 with 10% lactic acid in 10 mL of milk. Next, 0.1 mL (1%) of GODO-TCF and 0.9 mL, or 1 mL (10%) of sterile water was added to the 10 mL of milk, and the resultant preparation was left to stand at 43° C. for 60 minutes. Meanwhile, as a comparative example, in order to inactivate the protease activity in GODO-TCF, GODO-TCF was diluted by 4 times with distilled water, and then the diluted preparation was subjected to heat treatment at 100° C. for 120 minutes. Into milk of which the pH had been adjusted to 5.9, 0.4 mL (1% in terms of before dilution) of GODO-TCF to which heat treatment had been performed after dilution, and 0.6 mL of sterile water were added.

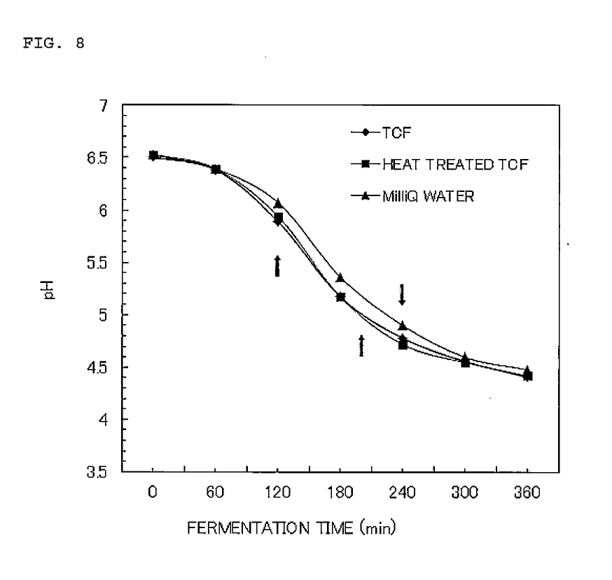

The results are shown in FIG. 7. As shown in FIG. 7, in a case where GODO-TCF in which protease had been inactivated was added, milk was not coagulated, whereas the milk to which 1% and 10% of GODO-TCF had been added was coagulated.

### Example 2

10 g of milk (Oishii Gyunyu, manufactured by Meiji Co., Ltd.) was weighed out in a 15-mL assist tube, and 0.2 g (2%, w/w) of skim milk was added to the milk, and the resultant preparation was thoroughly stirred and dissolved, and was boiled for 5 minutes in boiling water to sterilize. Then, cooled milk was preincubated in 43° C., and 0.1 mL (1%, w/v) of TCF sterilized by filtration through a 0.2-μm filter, and 0.3 mL of sterilized MilliQ water were aseptically added in a clean bench. As a control, one obtained by adding 1.2 mL of the TCF diluted by 4 times with MilliQ water and boiled in boiling water for 2 hours, and one obtained by adding 1.2 mL of sterilized MilliQ water were prepared. Then, in a clean bench, 0.5 g (5%, w/w) of a starter (Bulgaria Yogurt, manufactured by Meiji Co., Ltd.) was aseptically added to each tube, and the resultant preparation was stirred gently by inverting the tube 2 to 3 times. Seven tubes were prepared for each sample, and the fermentation was stopped every one hour from immediately after the addition of a starter until 6 hours later, and the pH was measured.

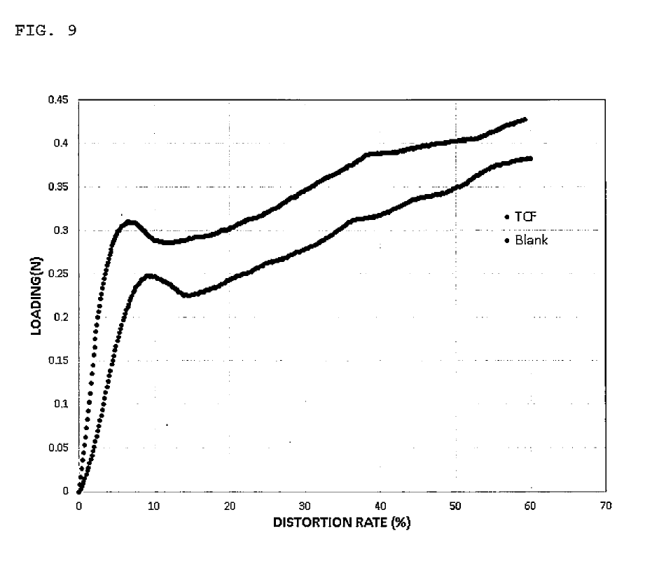

As a result, as shown in FIG. 8, it was found that in both controls of the heat-treated GODO-TCF (protease inactivated) and the MilliQ water addition, coagulation occurred in the vicinity of the isoelectric point (pl=4.6) of the casein protein in milk, whereas in a case of the GODO-TCF addition, the coagulation occurred in the vicinity of pH 5.9.

### Example 3

### (Method)

55.8 g of milk (Oishii Gyunyu, manufactured by Meiji Co., Ltd.) was weighed out in a jam bottle (Jam 90ST, manufactured by Toyo Glass Co., Ltd.), and 1.2 g (2%, w/w) of skim milk was added to the milk, and the resultant preparation was thoroughly stirred and dissolved, and was boiled for 5 minutes in boiling water to sterilize. Then, cooled milk was preincubated at 43° C., and 6 mL (1%, w/v) of the GODO-TCF sterilizedbyfiltrationthrough a filter having a pore size of 0.2 μm was aseptically added in a clean bench. As the blank, 0.6 mL (1%) of a 50 mM citrate buffer solution (pH 5.2, r.t.) was added. Then, 3 g (5%, w/w) of a starter (Bulgaria Yogurt, manufactured by Meiji Co., Ltd.) was aseptically added to each tube in a clean bench, the resultant preparation was stirred gently by inverting the tube 2 to 3 times, and the fermentation was conducted at 43° C. for 4 hours. The fermented milk samples were subjected to texture analysis in each container.

For the analyzer, a creep meter RE-33005C (YAMADEN co., ltd.) was used. In the breaking strength analysis, the measurement was performed using a jig No. 3 under the conditions of 4° C., an amplifier magnification of 10, a storage pitch of 750, a measured strain rate of 60%, a measurement speed of 10 mm/sec, and automatic measurement of sample thickness. In the texture analysis under a non-breaking condition, the measurement was performed using a jig No. 3 under the conditions of 4° C., an amplifier magnification of 10, a storage pitch of 875, a measured strain rate of 5%, a measurement speed of 3 mm/sec, a return distance of 30 mm, and automatic measurement of sample thickness. In the texture analysis under a breaking condition, the measurement was performed using a jig No. 3 under the conditions of 4° C., an amplifier magnification of 10, a storage pitch of 875, a measured strain rate of 50%, a measurement speed of 10 mm/sec, a return distance of 30 mm, and automatic measurement of sample thickness.

<Data Obtained from the Texture Profile (Two-Bite Method)>

Hardness (H): The maximum load on application. For semi-solid food, stress divided by jig cross-sectional area.

Brittleness (B): The amount of juice drop at the time of the occurrence of partial destruction on compression.

Adhesive force (C): The maximum load value at which adhesion at the time of the return tension after compression is observed. For semi-solid food, the jig cross-sectional area is taken into account.

Adherability (A3): The amount difficulty in separation in a case of having adhesiveness.

Cohesiveness (A2/A1): The ratio and the deformation amount of compression energy between the first stroke and the second stroke.

Elasticity (T2/T1): The ratio in the change amount reaching the maximum peak between the first stroke and the second stroke.

Gum property (hardness×cohesiveness): Higher value indicates higher hardness, and it can be assumed to be rubbery.

Chewiness (hardness×cohesiveness×elasticity): Higher value indicates that more energy is required for chewing.

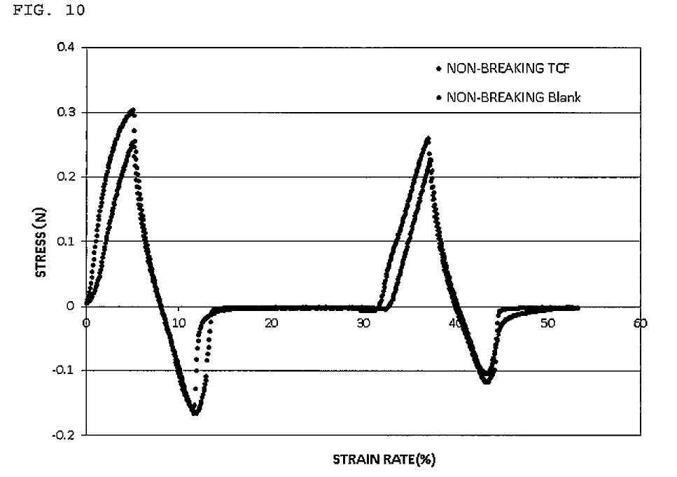

In the breaking strength analysis, in the GODO-TCF added sample, the breaking load was increased by 0.03 N and the breaking strain rate was decreased by 4.4% as compared with those of the additive-free sample at a breaking point (FIG. 9, and Table 1).

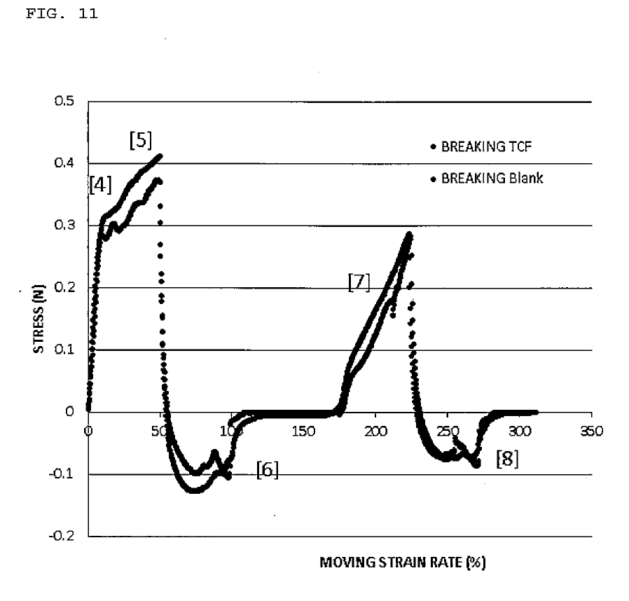

In the texture analysis of non-breaking (FIG. 10), the maximum stress during surface compression ([1] and [3]) was increased by 0.5 N in the GODO-TCF added sample. As to the cohesiveness, and the adherability ([2]), the values were decreased in the GODO-TCF added sample.

In the texture analysis of breaking (FIG. 11), in the GODO-TCF added sample, the stress at the time of breaking was increased ([4]). Waveforms ([5] and [7]) showing the breaking stress inside the curd are relatively smooth in the GODO-TCF added sample, whereas in the additive-free sample, a breaking point was able to be confirmed in several places. Moreover, in the cohesiveness and the adherability ([6] and [8]), the values were higher in the GODO-TCF added sample as compared with those in the additive-free sample. In the waveforms of the adherability ([6] and [8]) at the time of being pulled out, it was found that the GODO-TCF added sample was smoothly pulled out, whereas in the additive-free sample, the waveform was disturbed, and resistance was applied.

TABLE 1
Measurement values of breaking strength analysis

| | Breaking load | Breaking energy | Breaking strain rate |
|---|---|---|---|
| | (N) | (J/m³) | (%) |
| TCF added | 0.316 | 375.47 | 6.64 |
| Blank | 0.282 | 291.32 | 11.2 |

### Example 4

### (Method)

In accordance with the fermented milk preparation method of Example 3, fermented milk samples were prepared on a 200 g scale. As the Sumizyme C (cellulase preparation, manufactured by Shin-Nihon Chemical Co., Ltd.), a preparation diluted so as to have chymosin activity equivalent to that of GODO-TCF by the chymosin activity method described in (3) of Production Example 1 was used. For the Blank, 0.6 mL (1%) of a 50 mM citrate buffer solution (pH 5.2, r.t.) was added.

As the analyzer, a taste sensing device TS-5000Z (Insent) was used. For the sample for taste sensor analysis, one obtained by diluting a fermented milk sample by 2 times with purified water (w/w) was used. As a control for analysis, one obtained by similarly diluting Bulgaria Yogurt (Meiji Co., Ltd.) was used. For the sensor used for analysis, a blend membrane, a plus membranes, a minus membrane, and GL1 (sweet taste sensor) were used.

The results of the taste sensor analysis are shown in Table 2. Each analysis value is expressed as a relative value based on the analysis values of Bulgaria Yogurt.

In the 1% GODO-TCF added sample, sweetness, and acidic bitterness were lower than those in the samples of control, 0.1% GODO-TCF, citrate buffer solution added. In the 0.1% GODO-TCF added sample, the results were almost the same as those of the citrate buffer solution added sample. In the Sumizyme C added sample, acidic bitterness, and a aftertaste from acidic bitterness were higher.

From the results described above, it was found that when the influence of salts and buffer components was removed, changes in tastes other than the acidic bitterness and sweetness were not generated. In addition, it was found that bitterness was hardly generated even when the GODO-TCF derived protease was allowed to act on a milk protein.

TABLE 2
Analysis values of each taste by taste sensor

| | | | | | Aftertaste | | | | |
|---|---|---|---|---|---|---|---|---|---|
| | | | | | from | Aftertaste | | | |
| | | Acidic | | Mineral-based | acidic | from | | | |
| Sample | Sourness | bitterness | Astringency | bitter taste 2 | bitterness | astringency | Umami | Richness | Sweetness |
| Bulgaria | 0 | 0 | 0 | 0 | 0 | 0 | 0 | 0 | 0 |
| (Control) | | | | | | | | | |
| 1% TCF | −0.09 | −0.32 | 0.09 | 0.05 | 0 | 0.14 | −0.03 | 0.56 | −0.41 |
| 0.1% TCF | 0.05 | −0.03 | −0.09 | 0.06 | 0.01 | 0.04 | −0.02 | 0.35 | 0.11 |
| Citrate | −0.14 | 0.12 | 0 | 0.09 | 0.07 | 0.05 | 0.03 | 0.48 | 0.19 |
| buffer | | | | | | | | | |
| solution | | | | | | | | | |
| (Blank) | | | | | | | | | |
| Sumizyme | −0.41 | 0.19 | 0.13 | 0.12 | 0.27 | 0.14 | 0.17 | 0.74 | −0.04 |

1. A dairy product, comprising:
a milk-derived raw material comprising casein; and
a protein consisting of an amino acid sequence having 90% or more identity to the amino acid sequence represented by SEQ ID NO: 1.

2. The dairy product according to claim 1, wherein the protein is a protein derived from a microorganism belonging to the genus Trichoderma.

3. The dairy product according to claim 1, further comprising:
lactic acid bacteria or yeast.

4. The dairy product according to claim 1, wherein the dairy product is fermented milk or cheese.

5. The dairy product according to claim 1, wherein the dairy product has a breaking stress in a two-bite method of 5,500 Pa or more, and a breaking adhesion of 800 J/m³ or more.

6. The dairy product according to claim 1, substantially not comprising:
a coagulation component selected from the group consisting of a thickening agent, a stabilizer, a gelling agent, and an adhesive paste.

7. A method for producing a dairy product, the method comprising:
adding a protein consisting of an amino acid sequence having 90% or more identity to the amino acid sequence represented by SEQ ID NO: 1, and lactic acid bacteria to a milk-derived raw material comprising casein; and
fermenting a mixture of the protein and the lactic acid bacteria with the milk-derived raw material.

8. A milk-coagulating agent for a dairy product, comprising:
a protein consisting of an amino acid sequence having 90% or more identity to the amino acid sequence represented by SEQ ID NO: 1.


SEQUENCE LISTING (5 sequences)

SEQ ID NO: 1 - protein, 395 aa, Trichoderma reesei
MKSALLAAAALVGSAQAGIHKMKLQKVSLEQQLEGSSIEAHVQQLGQKYMGVRPTSRAEVMFNDKPPKVQGGHPVPVTNFMNAQYFSEITIGTPPQSFKVVLDTGSSNLWVPSQSCNSIACFLHSTYDSSSSSTYKPNGSDFEIHYGSGSLTGFISNDVVTIGDLKIKGQDFAEATSEPGLAFAFGRFDGILGLGYDTISVNGIVPPFYQMVNQKLIDEPVFAFYLGSSDEGSEAVFGGVDDAHYEGKIEYIPLRRKAYWEVDLDSIAFGDEVAELENTGAILDTGTSLNVLPSGLAELLNAEIGAKKGFGGQYTVDCSKRDSLPDITFSLAGSKYSLPASDYIIEMSGNCISSFQGMDFPEPVGPLVILGDAFLRRYYSVYDLGRDAVGLAKAK

SEQ ID NO: 2 - protein, 395 aa, Trichoderma virens Gv29-8
MKSALIAAAALVGSAQAGVHKMKLQKVSLEQQLEGSTIESQVQHLGQKYMGVRPTSRADVMFNDKLPKIQGGHPVPVTNFMNAQYFSEITIGTPPQTFKVVLDTGSSNLWVPSQSCNSIACFLHATYDSSSSSTYKQNGSDFEIHYGSGSLTGFISNDVVTIGDLKIQKQDFAEATSEPGLAFAFGRFDGILGLGYDTISVNGIIPPFYQMVNQKLLDEPVFAFYLGSGDEGSEAVFGGVDESHYSGKIEYIPLRRKAYWEVDLDSIAFGDEVAELENTGAILDTGTSLNVLPSGLAELLNAEIGAKKGFGGQYTVDCSKRDSLPDITFSLAGSKYSLPATDYIIEMSGNCISSFQGMDFPEPVGPLVILGDAFLRRYYSVYDLGKNAVGLAKAK

SEQ ID NO: 3 - protein, 395 aa, Trichoderma atroviride
MKSALIAAAALVGSAQAGVHKMKLQKVSLEQQLEGSSIEAQVQQLGQKYMGVRPTSRVDVMFNDNVPKVKGGHPVPVTNFMNAQYFSEITIGSPPQTFKVVLDTGSSNLWVPSQSCNSIACFLHSTYDSSSSSSYKKNGSDFEIHYGSGSLTGFISNDVVTIGDLQIKGQDFAEATSEPGLAFAFGRFDGILGLGYDTISVNGIVPPFYQMVNQKLLDEPVFAFYLGSGDEGSVATFGGVDESHYSGKIEYIPLRRKAYWEVDLDSIAFGDEVAELENTGAILDTGTSLNVLPSGIAELLNAEIGAKKGYGGQYTIDCAKRDSLPDITFSLAGSKYSLPASDYILEVSGSCISTFQGMDFPEPVGPLVILGDAFLRRYYSVYDLGKGAVGLAKAK

SEQ ID NO: 4 - DNA, 28 bp, Artificial Sequence
  Designed primer based on Trichoderma reesei
tacaaagaggctggttcctagcttcgtc

SEQ ID NO: 5 - DNA, 27 bp, Artificial Sequence
  Designed primer based on Trichoderma reesei
taggactgtcttccgctctcaaactgc